(19) United States
(12) Patent Application Publication
(10) Pub. No.: US 2016/0129134 A1
(43) Pub. Date: May 12, 2016

(54) COMPOSITIONS COMPRISING CROSS-LINKED HYALURONIC ACID AND CYCLODEXTRIN

(71) Applicant: GALDERMA S.A., Cham (CH)

(72) Inventor: Jean-Guy BOITEAU, Valbonne (FR)

(73) Assignee: Galderma S.A., CHAM, OT (CH)

(21) Appl. No.: 14/898,299

(22) PCT Filed: Jun. 9, 2014
(86) PCT No.: PCT/EP2014/061942

§ 371 Date: Dec. 14, 2015

Related U.S. Application Data
(60) Provisional application No. 61/834,952, filed on Jun. 14, 2013.

(51) Int. Cl. A61K 47/48; A61K 8/34; C08B 37/08; A61K 8/73; C08B 37/16; A61K 31/07; A61Q 19/00

(52) U.S. Cl. CPC A61K 47/48969; A61K 31/07; A61K 8/342; A61Q 19/00; A61K 8/738; A61K 8/735; C08B 37/0015; C08B 37/0072; A61K 2800/10

(57) ABSTRACT

The present invention relates to a hyaluronic acid composition comprising a hyaluronic acid and one or more cyclodextrin molecules covalently bound to said hyaluronic acid via a bi- or polyfunctional crosslinking agent, wherein the covalent bonds between said hyaluronic acid and said crosslinking agent and between said crosslinking agent and said cyclodextrin molecules are ether bonds. The present invention relates to medical and cosmetic (non-medical) uses of such compositions further comprising a pharmaceutical or medical agent and to a method of preparing a slow release formulation.

### FIELD OF THE INVENTION

The present invention relates to the field of hyaluronic acid compositions and the use of such compositions in medical and/or cosmetic applications.

### BACKGROUND

One of the most widely used biocompatible polymers for medical use is hyaluronic acid (HA). It is a naturally occurring polysaccharide belonging to the group of glycosaminoglycans (GAGs). Hyaluronic acid and the other GAGs are negatively charged heteropolysaccharide chains which have a capacity to absorb large amounts of water. Hyaluronic acid and products derived from hyaluronic acid are widely used in the biomedical and cosmetic fields, for instance during viscosurgery and as a dermal filler.

Water-absorbing gels, or hydrogels, are widely used in the biomedical field. They are generally prepared by chemical crosslinking of polymers to infinite networks. While native hyaluronic acid and certain crosslinked hyaluronic acid products absorb water until they are completely dissolved, crosslinked hyaluronic acid gels typically absorb a certain amount of water until they are saturated, i.e. they have a finite liquid retention capacity, or swelling degree.

Since hyaluronic acid is present with identical chemical structure except for its molecular mass in most living organisms, it gives a minimum of reactions and allows for advanced medical uses. Crosslinking and/or other modifications of the hyaluronic acid molecule is necessary to improve its duration in vivo. Furthermore, such modifications affect the liquid retention capacity of the hyaluronic acid molecule. As a consequence thereof, hyaluronic acid has been the subject of many modification attempts.

Cyclodextrins (sometimes called cycloamyloses), also referred to herein as CDs, are a family of compounds made up of sugar molecules bound together in a ring (cyclic oligosaccharides). Cyclodextrins are produced from starch by means of enzymatic conversion. Typically, cyclodextrins are constituted by 6-8 glucopyranoside units, and have a structural conformation resembling toroids with the primary hydroxyl groups of the glucopyranoside units arranged along the smaller opening of the toroid and the secondary hydroxyl groups of the glucopyranoside units arranged along the larger opening of the toroid. Because of this arrangement, the interior of the toroids is considerably less hydrophilic than the aqueous environment and thus able to host other hydrophobic molecules. In contrast, the exterior is sufficiently hydrophilic to impart cyclodextrins (or their complexes) water solubility.

When a hydrophobic molecule (the guest) is contained, fully or partially, within the interior of the cyclodextrin (the host), this is referred to as an inclusion complex or guest/host complex. The formation of the guest/host complex can greatly modify the physical and chemical properties of the guest molecule, mostly in terms of water solubility. This is a reason why cyclodextrins have attracted much interest in pharmaceutical applications: because inclusion compounds of cyclodextrins with hydrophobic molecules are able to penetrate body tissues, these can be used to release biologically active compounds under specific conditions. In most cases the mechanism of controlled degradation of such complexes is based on change of pH, leading to the cleavage of hydrogen or ionic bonds between the host and the guest molecules. Other mechanisms for the disruption of the complexes include heating or action of enzymes able to cleave α-1,4 linkages between glucose monomers.

### DESCRIPTION OF THE INVENTION

An object of the present invention is to provide improved formulations for administration of pharmaceutical and/or cosmetic substances.

According to aspects illustrated herein, there is provided a hyaluronic acid composition comprising

a hyaluronic acid and

one or more cyclodextrin molecules covalently bound to said hyaluronic acid via a bi- or polyfunctional crosslinking agent,

wherein the covalent bonds between said hyaluronic acid and said crosslinking agent and between said crosslinking agent and said cyclodextrin molecules are ether bonds.

The cyclodextrin molecules are used as carriers (hosts) for a pharmaceutical agent (guest). When a pharmaceutical agent (the guest) is contained, fully or partially, within the interior of the cyclodextrin (the host), this is referred to as an inclusion complex or guest/host complex. The cyclodextrin may then release the pharmaceutical agent under specific conditions, e.g. due to change in pH leading to the cleavage of hydrogen or ionic bonds between the host and the guest molecules.

The cyclodextrin molecules are attached to the hyaluronic acid in order to reduce migration of the cyclodextrin (or guest/host complex) form the site of administration, e.g. injection. This way the site of release of the pharmaceutical agent from the cyclodextrin can be controlled.

Also, in order to increase temporal control of the release of the pharmaceutical agent, it has been found that the influence of cleavage of the bonds between the cyclodextrin (or guest/host complex) and the hyaluronic acid should be minimized. In other words, it is desired that the release of the pharmaceutical agent is, as far as possible dependent on the physical release from the cyclodextrin rather than on chemical degradation.

In the disclosed compositions, the cyclodextrin molecules are attached to the hyaluronic acid by ether bonds. The use of ether bonds in the cyclodextrin-hyaluronic acid linkage has been found to be advantageous compared to, e.g., ester bonds, since the ether bond is more stable to degradation in vivo.

The use of a less stable bond between the hyaluronic acid and cyclodextrin molecules could lead to premature loss of cyclodextrin (or guest/host complex) from the site of injection.

The cyclodextrin of the hyaluronic acid composition may in practice be any cyclodextrin capable of acting as the host molecule in a guest/host complex together with a pharmaceutical agent. Cyclodextrins may generally be constituted by 5-32 glucopyranoside units. However, cyclodextrins constituted by 6-8 glucopyranoside units are generally preferred for the formation of guest/host complexes with pharmaceutical agents. Cyclodextrins constituted by 6, 7 and 8 glucopyranoside units are often referred to as α-, β- and γ-cyclodextrins respectively.

According to an embodiment, the cyclodextrin molecules are constituted by 6 glucopyranoside units (α-cyclodextrin).

According to an embodiment, the cyclodextrin molecules are constituted by 7 glucopyranoside units (β-cyclodextrin).

According to an embodiment, the cyclodextrin molecules are constituted by 8 glucopyranoside units (γ-cyclodextrin).

Cyclodextrins are often chemically modified in order to improve their solubility in water and/or to optimize their performance in a specific application. The term cyclodextrin, α-cyclodextrin, β-cyclodextrin and γ-cyclodextrin, as used herein is also intended to encompass the functionally equivalent variants or derivatives thereof. Examples of such chemically modified cyclodextrins include, but are not limited to, hydroxypropyl and methyl cyclodextrins.

Examples of modified α-cyclodextrins for use with the hyaluronic acid composition include, but are not limited to, hydroxypropyl a cyclodextrin.

Examples of modified β-cyclodextrins for use with the hyaluronic acid composition include, but are not limited to, hydroxypropyl-β-cyclodextrin; 2,6-di-O-methyl-β-cyclodextrin; 6-O-maltosyl-β-cyclodextrin; 2-hydroxypropyl-β-cyclodextrin; methyl-β-cyclodextrin; sulfobutyl-β-cyclodextrin; monochlorotriazinyl-β-cyclodextrin; heptakis (2-ω-amino-O-oligo (ethylene oxide)-6-hexylthio)-β-cyclodextrin; ethylenediamino or diethylenetriamino bridged bis(β cyclodextrin)s; randomly methylated β-cyclodextrin; sulfobutyl ether-β-cyclodextrin; and monochlorotriazinyl-β-cyclodextrin.

Examples of modified γ-cyclodextrins for use with the hyaluronic acid composition include, but are not limited to, γ-cyclodextrin C6, and 2,3-di-O-hexanoyl-γ cyclodextrin. Further additional modified cyclodextrins are also shown in Tables 1-3 herein.

The bi- or polyfunctional crosslinking agent of the hyaluronic acid composition connects the cyclodextrin molecules to the hyaluronic acid. The bi- or polyfunctional crosslinking agent further acts as a spacer between the cyclodextrin molecules and the hyaluronic acid.

The bi- or polyfunctional crosslinking agent comprises two or more functional groups capable of reacting with functional groups of the hyaluronic acid and cyclodextrin molecules respectively, resulting in the formation of ether bonds. The bi- or polyfunctional crosslinking agent may for example selected from the group consisting of divinyl sulfone, multiepoxides and diepoxides.

According to an embodiment, the bi- or polyfunctional crosslinking agent comprises two or more glycidyl ether functional groups. The glycidyl ether functional groups react with primary hydroxyl groups of the hyaluronic acid and cyclodextrin molecules respectively, resulting in the formation of ether bonds.

According to embodiments the bi- or polyfunctional crosslinking agent is selected from the group consisting of 1,4-butanediol diglycidyl ether (BDDE), 1,2-ethanediol diglycidyl ether (EDDE) and diepoxyoctane.

According to a preferred embodiment the bi- or polyfunctional crosslinking agent is 1,4-butanediol diglycidyl ether (BDDE). BDDE reacts with the primary hydroxyl groups of a hyaluronan repeating unit and a cyclodextrin glucopyranoside unit resulting in the formation of two ether bonds.

According to an embodiment, the bi- or polyfunctional crosslinking agent for connecting the cyclodextrin molecules to the hyaluronic acid is the same as the crosslinking agent used for crosslinking the hyaluronic acid. According to a preferred embodiment 1,4-Butanediol diglycidyl ether (BDDE) is used both for crosslinking the hyaluronic acid and for connecting the cyclodextrin molecules to the hyaluronic acid.

The degree of substitution of the hyaluronic acid (number of cyclodextrin molecules per total number of hyaluronan repeating unit in the hyaluronic acid) is preferably in the range of between 0.5 and 50%, more preferably between 2 and 20%.

The hyaluronic acid composition is preferably aqueous and the hyaluronic acid and the cyclodextrins are preferably swelled, dissolved or dispersed in the aqueous phase.

The hyaluronic acid composition comprises a hyaluronic acid. The hyaluronic acid may be a modified, e.g. branched or crosslinked, hyaluronic acid. According to certain embodiments the hyaluronic acid is a crosslinked hyaluronic acid. According to specific embodiments the hyaluronic acid is a hyaluronic acid gel. The composition is preferably injectable.

Unless otherwise provided, the term “hyaluronic acid” encompasses all variants and combinations of variants of hyaluronic acid, hyaluronate or hyaluronan, of various chain lengths and charge states, as well as with various chemical modifications, including crosslinking. That is, the term also encompasses the various hyaluronate salts of hyaluronic acid with various counter ions, such as sodium hyaluronate. Various modifications of the hyaluronic acid are also encompassed by the term, such as oxidation, e.g. oxidation of —CH₂OH groups to —CHO and/or —COOH; periodate oxidation of vicinal hydroxyl groups, optionally followed by reduction, e.g. reduction of —CHO to —CH₂OH or coupling with amines to form imines followed by reduction to secondary amines; sulphation; deamidation, optionally followed by deamination or amide formation with new acids; esterification; crosslinking; substitutions with various compounds, e.g. using a crosslinking agent or a carbodiimide assisted coupling; including coupling of different molecules, such as proteins, peptides and active drug components, to hyaluronic acid; and deacetylation. Other examples of modifications are isourea, hydrazide, bromocyan, monoepoxide and monosulfone couplings.

The hyaluronic acid can be obtained from various sources of animal and non-animal origin. Sources of non-animal origin include yeast and preferably bacteria. The molecular weight of a single hyaluronic acid molecule is typically in the range of 0.1-10 MDa, but other molecular weights are possible.

In certain embodiments the concentration of said hyaluronic acid is in the range of 1 to 100 mg/ml. In some embodiments the concentration of said hyaluronic acid is in the range of 2 to 50 mg/ml. In specific embodiments the concentration of said hyaluronic acid is in the range of 5 to 30 mg/ml or in the range of 10 to 30 mg/ml. In certain embodiments, the hyaluronic acid is crosslinked. Crosslinked hyaluronic acid comprises crosslinks between the hyaluronic acid chains, which creates a continuous network of hyaluronic acid molecules which is held together by the covalent crosslinks, physical entangling of the hyaluronic acid chains and various interactions, such as electrostatic interactions, hydrogen bonding and van der Waals forces.

Crosslinking of the hyaluronic acid may be achieved by modification with a chemical crosslinking agent. The chemical crosslinking agent may for example selected from the group consisting of divinyl sulfone, multiepoxides and diepoxides. According to an embodiment, the hyaluronic acid is crosslinked by a bi- or polyfunctional crosslinking agent comprising two or more glycidyl ether functional groups. According to embodiments the chemical crosslinking agent is selected from the group consisting of 1,4-butanediol diglycidyl ether (BDDE), 1,2-ethanediol diglycidyl ether (EDDE) and diepoxyoctane. According to a preferred embodiment, the chemical crosslinking agent is 1,4-butanediol diglycidyl ether (BDDE).

The crosslinked hyaluronic acid product is preferably biocompatible. This implies that no, or only very mild, immune response occurs in the treated individual. That is, no or only very mild undesirable local or systemic effects occur in the treated individual.

The crosslinked hyaluronic acid product according to the invention may be a gel, or a hydrogel. That is, it can be regarded as a water-insoluble, but substantially dilute crosslinked system of hyaluronic acid molecules when subjected to a liquid, typically an aqueous liquid.

The gel contains mostly liquid by weight and can e.g. contain 90-99.9% water, but it behaves like a solid due to a three-dimensional crosslinked hyaluronic acid network within the liquid. Due to its significant liquid content, the gel is structurally flexible and similar to natural tissue, which makes it very useful as a scaffold in tissue engineering and for tissue augmentation.

As mentioned, crosslinking of hyaluronic acid to form the crosslinked hyaluronic acid gel may for example be achieved by modification with a chemical crosslinking agent, for example BDDE (1,4-butandiol diglycidylether). The hyaluronic acid concentration and the extent of crosslinking affects the mechanical properties, e.g. the elastic modulus G′, and stability properties of the gel. Crosslinked hyaluronic acid gels are often characterized in terms of “degree of modification”. The degree of modification of hyaluronic acid gels generally range between 0.1 and 15 mole %. The degree of modification (mole %) describes the amount of crosslinking agent(s) that is bound to HA, i.e. molar amount of bound crosslinking agent(s) relative to the total molar amount of repeating HA disaccharide units. The degree of modification reflects to what degree the HA has been chemically modified by the crosslinking agent. Reaction conditions for crosslinking and suitable analytical techniques for determining the degree of modification are all well known to the person skilled in the art, who easily can adjust these and other relevant factors and thereby provide suitable conditions to obtain a degree of modification in the range of 0.1-2% and verify the resulting product characteristics with respect to the degree of modification. A BDDE (1,4-butandiol diglycidylether) crosslinked hyaluronic acid gel may for example be prepared according to the method described in Examples 1 and 2 of published international patent application WO 9704012.

In a preferred embodiment the hyaluronic acid of the composition is present in the form of a crosslinked hyaluronic acid gel crosslinked by a chemical crosslinking agent, wherein the concentration of said hyaluronic acid is in the range of 10 to 30 mg/ml and the degree of modification with said chemical crosslinking agent is in the range of 0.1 to 2 mole %.

Hyaluronic acid gels may also comprise a portion of hyaluronic acid which is not crosslinked, i.e not bound to the three-dimensional crosslinked hyaluronic acid network. However, it is preferred that at least 50% by weight, preferably at least 60% by weight, more preferably at least 70% by weight, and most preferably at least 80% by weight, of the hyaluronic acid in a gel composition form part of the crosslinked hyaluronic acid network.

Hyaluronic acid compositions as described herein may advantageously be used for the transport or administration and slow or controlled release of various pharmaceutical or cosmetic substances. The composition is preferably injectable.

According to an embodiment, the hyaluronic acid composition further comprises a guest molecule forming a guest-host complex with at least one of said cyclodextrin molecules. The guest molecule may for example be a pharmaceutical agent or a cosmetic agent. According to an embodiment, the guest molecule is a pharmaceutical agent. According to an embodiment, the guest molecule is a cosmetic agent. According to an embodiment, the guest molecule is retinol. The guest molecule is generally hydrophobic or lipophilic or has a portion/moiety which is hydrophobic or lipophilic.

The size and properties of the guest molecule determines which cyclodextrin is suitable as host. Much effort has been invested in the scientific field to determine suitable cyclodextrin host molecules for various pharmaceutical guest molecules. Some of the guest-host complexes identified are presented in Tables 1-3 herein.

The guest molecule may be complexed with the cyclodextrin host molecule before or after the cyclodextrin molecule is covalently attached to the hyaluronic acid, however in some cases it may be preferable

According to aspects illustrated herein, there is provided a hyaluronic acid composition comprising a pharmaceutical agent as described herein, for use as a medicament.

According to aspects illustrated herein, there is provided a hyaluronic acid composition comprising a pharmaceutical agent as described herein for use in the treatment of a condition susceptible to treatment by said pharmaceutical agent.

According to aspects illustrated herein, there is provided the use of a hyaluronic acid composition comprising a pharmaceutical agent as described herein, for the manufacture of a medicament for treatment of a condition susceptible to treatment by said pharmaceutical agent.

According to aspects illustrated herein, there is provided a method of treating a patient suffering from a condition susceptible to treatment by a pharmaceutical agent by administering to the patient a therapeutically effective amount of a hyaluronic acid composition comprising said pharmaceutical agent as described herein.

According to aspects illustrated herein, there is provided a method of cosmetically treating skin, which comprises administering to the skin a hyaluronic acid composition as described herein comprising a cosmetic agent.

According to aspects illustrated herein, there is provided a method of preparing a slow release formulation of a guest molecule capable of forming a guest-host complex with a cyclodextrin molecule, comprising the steps:

a) providing a hyaluronic acid and one or more cyclodextrin molecules capable of forming a guest-host complex with the guest molecule,

b) covalently binding said cyclodextrin molecules to said hyaluronic acid using a bi- or polyfunctional crosslinking agent, wherein the covalent bonds formed between said hyaluronic acid and said crosslinking agent and between said crosslinking agent and said cyclodextrin molecules are ether bonds, and

c) bringing a solution of the guest molecule into contact with the cyclodextrin molecules bound to the hyaluronic acid under conditions allowing for the formation of a guest-host complex between the cyclodextrin molecules and the guest molecule, and optionally

d) recovering the guest-host complex bound to the hyaluronic acid.

According to an embodiment, said bi- or polyfunctional crosslinking agent comprises two or more glycidyl ether functional groups. In a preferred embodiment, said bi- or polyfunctional crosslinking agent is 1,4-butanediol diglycidyl ether (BDDE).

According to an embodiment, said guest molecule is a pharmaceutical agent. According to an embodiment, said guest molecule is a cosmetic agent.

According to an embodiment, said guest molecule is retinol.

Further, non-limiting examples of pharmaceutical agents and cyclodextrins capable of forming guest-host complexes are provided in tables 1-3.

TABLE 1
Compiled from A. Magnúsdóttir, M. Másson and T. Loftsson, J.
Incl. Phenom. Macrocycl. Chem. 44, 213-218, 2002

| Cyclodextrin type | Drugs |
|---|---|
| α-Cyclodextrin | Alprostadil (PGE1) |
| | Cefotiam hexetil HCl |
| β-Cyclodextrin | Benexate HCl |
| | Dexamethasone |
| | Iodine |
| | Nicotine |
| | Nimesulide |
| | Nitroglycerin |
| | Omeprazol |
| | PGE2 |
| | Piroxicam |
| | Tiaprofenic acid |
| 2-Hydroxypropyl-β-cyclodextrin | Cisapride |
| | Hydrocortisone |
| | Indomethacin |
| | Itraconazole |
| | Mitomycin I |
| Randomly methylated β-cyclodextrin | 17β-Estradiol |
| | Chloramphenicol |
| Sulfobutylether β-cyclodextrin | Voriconazole |
| | Ziprasidone maleate |
| 2-Hydroxypropyl-γ-cyclodextrin | Diclofenac sodium |

TABLE 2
Compiled from Amber Vyas, Shailendra Saraf, Swarnlata Saraf
J. Incl. Phenom. Macrocycl. Chem. (2008) 62: 23-42

| Cyclodextrin type | Drugs |
|---|---|
| β-CD, HP-β-CD | Ketoprofen |
| HP-β-CD, DM-β- | Gonadorelin, Leuprolide acetate, |
| CD, OM-β-CD | Recombinant human growth |
| | hormone, Lysozyme |
| β-CD, HP-β-CD | Niclosamide |
| β-CD | poly(propylene glycol) |
| | bisamine |
| β-CD | Dexamethasone, Flurbiprofen, |
| | Doxorubicin hydrochloride |
| 2-HP-β-CD | Glutathione |
| HP-α-CD, HP-β-CD | Triclosan, Furosemide |
| α-CD, β-CD, γ-CD | Insulin |
| β-CD, M-β-CD, HP-β- | Estradiol |
| CD, SB-β-CD | |
| γ-CDC6 | Progesterone |
| HP-β-CD | Nifedipine |
| HP-β-CD | Hydrocortisone |
| 2-HP-β-CD | Insulin |
| HP-β-CD | Carvedilol |
| HP-β-CD | Insulin |
| β-CD hydrate | Amlodipine |
| HP-β-CD | Methoxydibenzoylmethane |
| HP-β-CD | Insulin |
| β-CDMCT | Octyl methoxycinnamate |
| Heptakis-β-CD | TPPS |
| HP-β-CD | Saquinavir |
| β-CD, 2-HP-β-CD | Hydrocortisone, Progesterone |
| Bis-CD | Bovine serum albumin |
| HP-β-CD | Bovine serum albumin |
| a, b, γ-CD | Gabexate Mesylate |
| β-CDC6 | Tamoxifen citrate |
| HP-β-CD | Itraconazole |
| a, b, γ-CD | Indomethacin, Furosemide, |
| | Naproxen |
| β-CD, HP-β-CD | Nifedipine |
| β-CD | Amikacin |
| HP-β-CD, γ-CD, | Methacholine |
| RM-β-CD | |
| (SBE)7m-β-CD | Chlorpromazine hydrochloride |
| α-Cyclodextrin | Isotretinoin |
| MCT-β-CD | Miconazole |
| SBE7-β-CD | Carbamazepine |
| β-CD | Retinoic acid |
| HP-β-CD | Rh-interferon α-2a |
| β-cyclodextrin | Droepiandrosterone |
| β-CD, HP-β-CD, | Flurbiprofen |
| Me-β-CD | |
| β-CD | Naproxen, Ibuprofen |
| β-CD, Me-β-CD | Piroxicam |
| α-CD, β-CD, HP-β- | Melarsoprol |
| CD, RAME-β-CD | |
| HP-β-CD, PM-β-CD | Bupranolol |
| β-CD | Diclofenac |

TABLE 3
Compiled from R. Arun et al. Sci Pharm. 2008; 76; 567-598.

| Cyclodextrin type | Drugs |
|---|---|
| β-CD | Nimesulide, Sulfomethiazole, |
| | Lorazepam, Ketoprofen, |
| | Griseofulvin, Praziquantel, |
| | Chlorthalidon, Exodolac, |
| | Piroxicam, Itraconazole, Ibuprofen |
| α-CD | Praziquantel |
| γ-CD | Praziquantel, Omeprazole, Digoxin |
| HP-β-CD | Albendazole, DY-9760e, |
| | ETH-615, Levemopamil HCl, |
| | Sulfomethiazole, Ketoprofen, |
| | Griseofulvin, Itraconazole, |
| | Carbamazepine Zolpidem, |
| | Phenytoin, Rutin |
| DM-β-CD | Naproxen, Camptothesin |
| SBE-β-CD | DY-9760e, Danazol, Fluasterone, |
| | Spiranolactone |
| RM-β-CD | ETH-615, Tacrolimus |
| Randomly acetylated | Naproxen |
| amorphous-β-CD | |
| HP-β-CD, DM-β-CD | Promethazine |
| HP-β-CD | 2-ethylhexyl p- |
| | (dimethylamino)benzoate |
| β-CD | Glibenclamide |
| β-CD | Diclofenac sodium |
| β-CD, HP-β-CD | Quinaril |
| HP-β-CD, HP-γ-CD | Doxorubicin |
| HP-β-CD | Acyl ester prodrugs of |
| | Ganciclovir |
| γ-CD | Digoxin |
| HP-β-CD | Rutin |
| RDM-β-CD | Camptothesin |
| SBE-β-CD, HP-β-CD | Melphalan and |
| | Carmustine |
| γ-CD, HP-γ-CD, HP-β-CD | Paclitaxel |
| SBE-α-CD, SBE-β- | Spiranolactone |
| CD, HP-β-CD, γ-CD, β-CD | |
| β-CD | Flutamide |
| β-CD | Ketoprofen, Griseofulvin, |
| | Terfenadine |
| HP-β-CD | Albendazole, Ketoprofen, |
| | Phenytoin, Gliclazide |
| SBE7-β-CD | Spiranolactone |
| DM-β-CD | Tacrolimus |
| M-β-CD | Albendazole |
| ME-β-CD | Phenytoin |
| β-CD | Terfanidine, Tolbutamide |
| HP-β-CD | Tolbutamide, |
| | Amylobarbitone |
| HP-β-CD | Flutamide |
| γ-CD | Digoxin |
| HP-β-CD | Rutin |
| HP-β-CD | Clomipramine, |
| | Testosterone |
| SBE7-β-CD, | Danazole |
| HP-β-CD | |
| β-CD | Piroxicam |
| DM-β-CD | Carbamazepine |
| γ-CD | Digoxin |
| β-CD, SBE-β-CD | Glibenclamide |
| HP-β-CD | Miconazole |
| E-β-CD, Glu-β-CD, | Phenytoin |
| Mal-β-CD, SBE-β-CD, HP-β-CD | |
| β-CD, γ-CD, DM-β-CD, SBE-β-CD, | Spironolactone |
| HP-β-CD | |
| β-CD, HP-β-CD | Tolbutamide |
| DM-β-CD | α-Tocopheryl |
| | nicotinate |
| β-CD | Acyclovir |
| DM-β-CD, HP-β-CD | Diphenhydramine HCl |
| DM-β-CD | Cyclosporin A |

### Explanation of Abbreviations in Table 1-3

β-CD, Beta cyclodextrin; HP-β-CD, Hydroxypropyl beta cyclodextrin; DM-β-CD, 2,6-di-O-methyl beta cyclodextrin; OM-β-CD, 6-O-maltosyl beta cyclodextrin; 2HP-β-CD, 2-hydroxypropyl beta cyclodextrin; HP-α-CD, Hydroxypropyl alpha cyclodextrin; α-CD, Alpha cyclodextrin; γ-CD, Gamma cyclodextrin; M-β-CD, Methyl-β-cyclodextrin; SB-β-CD, Sulfobutyl beta cyclodextrin; γ-CDC6, Gamma cyclodextrin C6 or amphiphilic 2,3-di-O-hexanoyl gamma cyclodextrin; β-CDMCT, Monochlorotriazinyl beta cyclodextrin; Heptakis-β-CD, Heptakis (2-x-amino-O-oligo (ethylene oxide)-6-hexylthio) beta cyclodextrin; bis-CDs, Ethylenediamino or diethylenetriamino bridged bis(beta cyclodextrin)s; RMβ-CD, randomly methylated beta cyclodextrin; (SBE)7m-β-CD, Sulfobutyl ether-β-cyclodextrin; MCT-β-CD, Monochlorotriaziny beta cyclodextrin; Me-β-CD, Methyl beta cyclodextrin; SBE-β-CD, Sulfobutylether-β-cyclodextrin; TPPS, Anionic 5,10,15,20-tetrakis(4-sulfonatophenyl)-21 H,23H-porphyrin; E-β-CD, β-Cyclodextrin epichlorohydrin polymer; Glu-β-CD, Glucosyl-β-cyclodextrin; Mal-β-CD, Maltosyl-β-cyclodextrin.

### BRIEF DESCRIPTION OF THE DRAWINGS

The invention is further illustrated by FIGS. 1-3. FIGS. 1-3 represent exemplary embodiments only.

FIG. 1 is a schematic illustration of a hyaluronic acid composition comprising crosslinked hyaluronic acid, cyclodextrin molecules and a guest host complex between a cyclodextrinmolecule and a guest molecule (drug).

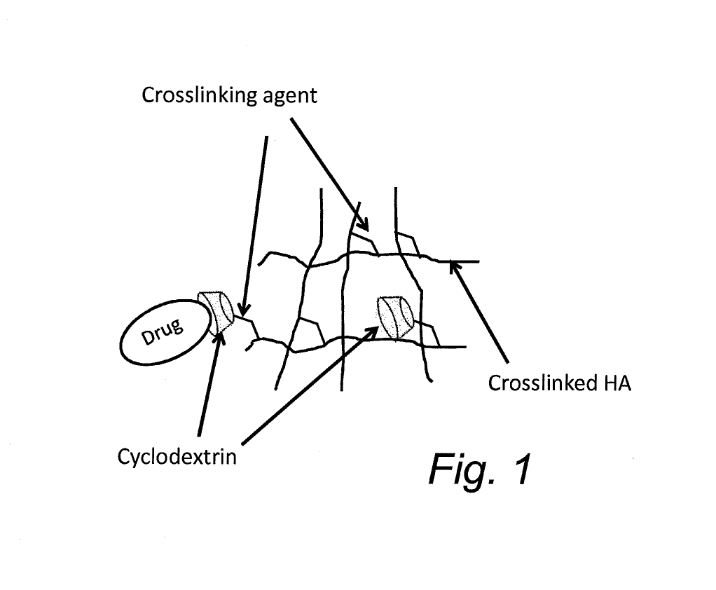

FIG. 2 depicts the chemical structures of cyclodextrins constituted by 6, 7 and 8 glucopyranoside units, also referred to as α-, β- and γ-cyclodextrins respectively.

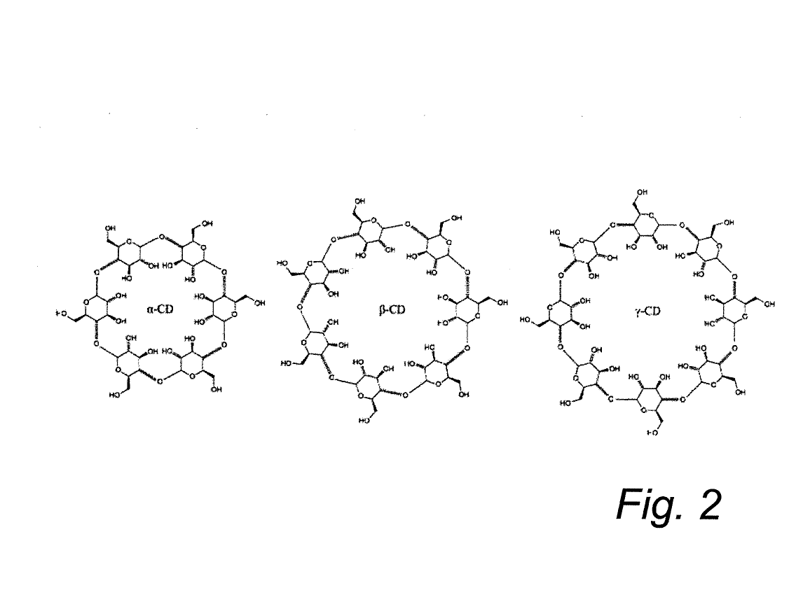

FIG. 3 is a schematic representation of the covalent binding of a cyclodextrin molecule to (BDDE crosslinked) hyaluronic acid (HA) using BDDE as a crosslinking agent, resulting in the formation of ether bonds between said hyaluronic acid and said crosslinking agent and between said crosslinking agent and said cyclodextrin molecule.

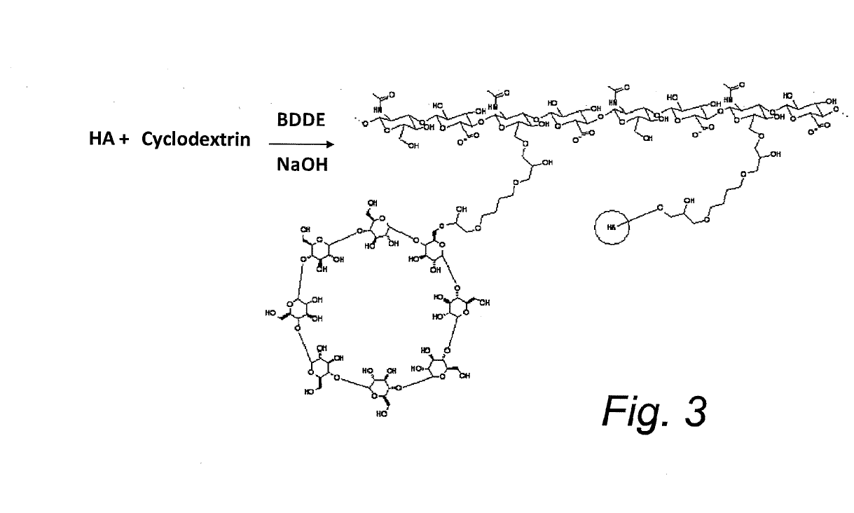

1. A hyaluronic acid composition comprising:
a hyaluronic acid and
one or more cyclodextrin molecules covalently bound to said hyaluronic acid via a bi- or polyfunctional crosslinking agent,
wherein the covalent bonds between said hyaluronic acid and said crosslinking agent and between said crosslinking agent and said cyclodextrin molecules are ether bonds.

2. A hyaluronic acid composition according to claim 1, wherein said cyclodextrin molecules are constituted by 5-32 glucopyranoside units.

3. A hyaluronic acid composition according to claim 2, wherein said cyclodextrin molecules are constituted by 6-8 glucopyranoside units.

4. A hyaluronic acid composition according to claim 3, wherein said cyclodextrin molecules are constituted by 6 glucopyranoside units (α-cyclodextrin).

5. A hyaluronic acid composition according to claim 3, wherein said cyclodextrin molecules are constituted by 7 glucopyranoside units (β-cyclodextrin).

6. A hyaluronic acid composition according to claim 3, wherein said cyclodextrin molecules are constituted by 8 glucopyranoside units (γ-cyclodextrin).

7. A hyaluronic acid composition according to claim 1, wherein said bi- or polyfunctional crosslinking agent comprises two or more glycidyl ether functional groups.

8. A hyaluronic acid composition according to claim 1, wherein said bi- or polyfunctional crosslinking agent is 1,4-Butanediol diglycidyl ether (BDDE).

9. A hyaluronic acid composition according to claim 1, wherein said hyaluronic acid is crosslinked hyaluronic acid.

10. A hyaluronic acid composition according to claim 9, wherein said hyaluronic acid is crosslinked by ether bonds.

11. A hyaluronic acid composition according to claim 10, wherein said hyaluronic acid is crosslinked by a bi- or polyfunctional crosslinking agent comprising two or more glycidyl ether functional groups.

12. A hyaluronic acid composition according to claim 11, wherein said hyaluronic acid is crosslinked by 1,4-Butanediol diglycidyl ether (BDDE).

13. A hyaluronic acid composition according to claim 1, wherein said hyaluronic acid is hyaluronic acid gel.

14. A hyaluronic acid composition according to claim 1, further comprising a guest molecule forming a guest-host complex with at least one of said cyclodextrin molecules.

15. A hyaluronic acid composition according to claim 14, wherein said guest molecule is a pharmaceutical agent.

16. A hyaluronic acid composition according to claim 14, wherein said guest molecule is a cosmetic agent.

17. A hyaluronic acid composition according to claim 14, wherein said guest molecule is retinol.

18. A hyaluronic acid composition according to claim 15 for use as a medicament.

19. A hyaluronic acid composition according to claim 15 for use in the treatment of a condition susceptible to treatment by said pharmaceutical agent.

20. Use of a hyaluronic acid composition according to claim 15, for the manufacture of a medicament for treatment of a condition susceptible to treatment by said pharmaceutical agent.

21. A method of treating a patient suffering from a condition susceptible to treatment by a pharmaceutical agent by administering to the patient a therapeutically effective amount of a hyaluronic acid composition according to claim 15 comprising said pharmaceutical agent.

22. A method of cosmetically treating skin, which comprises administering to the skin a hyaluronic acid composition according to claim 16.

23. A method of preparing a slow release formulation of a guest molecule capable of forming a guest-host complex with a cyclodextrin molecule, comprising the steps:
a) providing a hyaluronic acid and one or more cyclodextrin molecules capable of forming a guest-host complex with the guest molecule,
b) covalently binding said cyclodextrin molecules to said hyaluronic acid using a bi- or polyfunctional crosslinking agent, wherein the covalent bonds formed between said hyaluronic acid and said crosslinking agent and between said crosslinking agent and said cyclodextrin molecules are ether bonds, and
c) bringing a solution of the guest molecule into contact with the cyclodextrin molecules bound to the hyaluronic acid under conditions allowing for the formation of a guest-host complex between the cyclodextrin molecules and the guest molecule, and optionally
d) recovering the guest-host complex bound to the hyaluronic acid.

24. A method according to claim 23, wherein said bi- or polyfunctional crosslinking agent comprises two or more glycidyl ether functional groups.

25. A method according to claim 24, wherein said bi- or polyfunctional crosslinking agent is 1,4-Butanediol diglycidyl ether (BDDE).

26. A method according to claim 23, wherein said guest molecule is a pharmaceutical agent.

27. A method according to claim 23, wherein said guest molecule is a cosmetic agent.

28. A method according to claim 23, wherein said guest molecule is retinol.
